(19) United States
(12) Patent Application Publication
(10) Pub. No.: US 2026/0242349 A1
(43) Pub. Date: Aug. 20, 2026

(54) N,N'-SULFONIMIDAMIDES AS ANTICANCER, ANTIINFLAMMATORY, ANTIFIBROTIC AND NEUROPROTECTIVE AGENTS

(71) Applicant: Atux Iskay LLC, Plainsboro, NJ (US)

(72) Inventor: Michael Ohlmeyer, Plainsboro, NJ (US)

(21) Appl. No.: 19/159,289

(22) PCT Filed: Feb. 27, 2024
(86) PCT No.: PCT/US2024/017428

§ 371 Date: Aug. 22, 2025

Related U.S. Application Data
(60) Provisional application No. 63/448,620, filed on Feb. 27, 2023.

(51) Int. Cl. C07D 401/04; A61K 31/451; A61K 31/454; A61P 35/00; C07D 211/96

(52) U.S. Cl. CPC C07D 401/04; A61K 31/451; A61K 31/454; A61P 35/00; C07D 211/96

(57) ABSTRACT

A genus of N,N′-sulfonimidamides is disclosed. The compounds are of the following genus: Formula (i) or a pharmaceutically acceptable salt thereof. The compounds are useful in the treatment of a variety of disorders, including as a monotherapy in cancer treatment, or used in combination with other drugs to restore sensitivity to chemotherapy where resistance has developed.

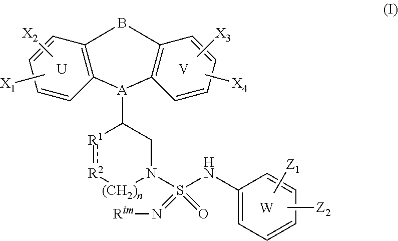

### RELATED APPLICATIONS

This application claims the benefit of U.S. Provisional Application No. 63/448,620 filed Feb. 27, 2023, which is incorporated herein by reference in its entirety.

### FIELD

This disclosure relates to the use of small molecule modulators of PP2A, comprising N,N′-sulfonimidamides to treat diseases such as cancer, inflammatory and autoimmune conditions, fibrotic and neurodegenerative diseases.

### BACKGROUND

Protein phosphatase 2A is one of the four major serine threonine phosphatases and is implicated in the negative control of cell growth and division. Protein phosphatase 2A holoenzymes are composed of a structural subunit A and a catalytic subunit C which form a catalytically competent PP2A AC heterodimer. PP2A AC heterodimers then associate with a regulatory B subunit, which controls substrate specificity. Regulatory B subunits occur in four families and their expression is cell-type and context dependent. The PP2A heterotrimeric protein phosphatase is a ubiquitous and conserved phosphatase with diverse cellular functions. Among the targets of PP2A of the classical B-subunit containing holoenzymes are proteins of oncogenic signaling cascades, such as Raf, MEK, and AKT, and in this role they function as tumor suppressors.

Recently PP2A AC heterodimers have been shown to interact directly with the INTS6 subunit of the Integrator Complex without any B subunit. Integrator associates with RNA Polymerase II (RNAPII) to control its transcriptional activity and PP2A AC associated Integrator acts to maintain RNAPII in a paused (i.e. transcriptionally stalled) state. This non-classical PP2A-Integrator complex thus functions to restrain transcription and this activity also contributes the tumor suppressor property of PP2A in transcriptionally addicted cancers, for example breast, lung, prostate, ovarian cancers, glioblastoma, melanoma, leukemia, and others in adults. Also in pediatric cancers such as neuroblastoma and medulloblastoma in children and infants.

Myc proteins (c-myc, Mycn and Mycl) target proliferative and apoptotic pathways vital for progression in cancer and it is overexpressed and deregulated in many human cancers. The control of Myc abundance through protein degradation has attracted considerable interest and Ser-62 phosphorylation by a number of kinases has been shown to stabilize the protein. PP2A is responsible for Ser-62 dephosphorylation which primes the protein for ubiquitylation and degradation, thus PP2A functions as a negative regulator of Myc.

The FOXO (Forkhead transcription factors, Class O) proteins are a group of transcription factors involved in control of a variety of physiological, metabolic and developmental pathways. They are downstream effectors in a number of signaling pathways including insulin and growth factor signaling: they are also regulated by oxidative stress and nutrient deprivation. Cellular processes affected by FOXO activity include cell cycle control, differentiation, proliferation, apoptosis and autophagy. Disregulation of FOXO mediated processes has been implicated in a number of pathologies including tumorigenesis, inflammation, fibrosis, diabetes, neurodegenerative and other age related conditions, amongst others. Activity of FOXO transcription factors are controlled in part by their sub-cellular localization, in particular their localization to the nucleus from the cytosol, and their subsequent transcriptional activation.

FOXO1 regulates expression of a number of genes that play critical roles in cell cycle and apoptosis. A pivotal regulatory mechanism of FOXO is reversible phosphorylation, catalyzed by kinases and phosphatases. Phosphorylation of FOXO1 is associated with 14-3-3 binding and cytosolic localization, whereas dephosphorylated FOXO1 translocates to the nucleus and is transcriptionally active. FOXO3 is regulated in an analogous manner.

PP2A interacts directly with FOXO1 and dephosphorylates FOXO1. Inhibition of PP2A phosphatases rescues FOXO1-mediated cell death by regulating the level of the pro-apoptotic protein BIM. In addition, PP2A directly regulates FOXO3a subcellular localization and transcriptional activation. Without wishing to be held to any particular theory, it may be that the compounds described herein promote apoptosis by acting on FOXO transcription factors via activation of PP2A.

Prostate cancer is the second leading cause of cancer death in men in America, behind lung cancer. According to the American Cancer Society, approximately 1 man in 36 will die of prostate cancer. Male hormones, specifically testosterone, fuel the growth of prostate cancer. By reducing the amount and activity of testosterone, the growth of advanced prostate cancer is slowed. Endocrine therapy, known as androgen ablation, is the first line of treatment for metastatic prostate cancer. Androgen deprivation therapy for metastatic prostate cancer results in tumor regression and symptomatic improvement in the majority of patients. However, metastatic prostate cancer inevitably progresses despite castrate levels of serum testosterone. Several new therapies have been approved for patients with castration-resistant prostate cancer (CRPC); however, none are curative and tumors ultimately develop resistance. To combat CRPC new approaches and novel therapies are required.

Breast cancer can affect both men and women. Breast cancer is the most prevalent cancer in women, after skin cancers, with about 1 in every 8 women expected to develop invasive breast cancer at some point. One subset of breast cancer expresses the androgen receptor (AR), which has been implicated as a therapeutic target in that subset. About 10-20% of breast cancers—more than one out of every 10—are found to be triple-negative. “Triple negative breast cancer” refers to a breast cancer that does not contain estrogen receptors, progesterone receptors, or human epidermal growth factor receptor 2 (HER2). This means that the growth of the cancer is not supported by the hormones estrogen and progesterone, nor by the presence of too many HER2 receptors. Therefore, triple-negative breast cancer does not respond to hormonal therapy (such as tamoxifen or aromatase inhibitors) or therapies that target HER2 receptors, such as Herceptin (chemical name: trastuzumab). While these tumors are often treatable, the chemotherapy is not targeted, and response durations are short. For doctors and researchers, there is intense interest in finding new medications that can treat breast cancer.

The compounds described herein, which are substituted N,N′-sulfonimidamides, exhibit anti-proliferative effects by increasing PP2A activity in cells with suppressed or deficient phosphatase activity and are therefore useful as monotherapy in cancer treatment. Additionally, they can be used in combination with other drugs to restore sensitivity to chemotherapy where resistance has developed. The compounds may also be used in treatment of other diseases characterized by deficient PP2A activity, for example lung fibrosis and Alzheimer's Disease.

### SUMMARY

A genus of substituted N,N′-sulfonimidamide and related compounds has now been found that modulate PP2A activity. The compounds deactivate pro-growth and pro-survival kinases such as phospo-ERK and phospho-AKT by promoting their deposphorylation by PP2A; they destabilize oncogenic MYC by promoting PP2A mediated dephoshorylation of MYC, and they promote RNAPII promoter proximal pausing during transcription. The compounds described herein exhibit anti-proliferative effects, and are useful in the treatment of a variety of disorders, including as a monotherapy in cancer treatment, or used in combination with other drugs to restore sensitivity to chemotherapy where resistance has developed.

In a first aspect the disclosure relates to compounds of formula (I):

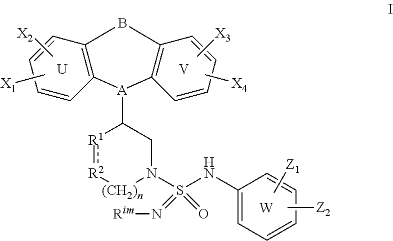

or a pharmaceutically acceptable salt thereof,
wherein:
   A is N or CH
   B is absent or is selected from a direct bond, —CH₂CH₂—, —CH═CH—, O, S, or —NR^{B}—C(O)—;
   R¹ is selected from CH₂ and may also be —O— or NR^{Q} when A is CH only;
   R² is selected from CH₂ or R¹ and R² taken together may for a carbon-carbon double bond;
   R^{im} is selected from H or lower alkyl
   U, V and W are independently cabocyclic aromatic or heteroaromatic rings;
   n=0 or 1;
   X₁, X₂, X₃ and X₄ are independently selected in each instance from hydrogen, halogen, nitro, cyano, (C₁-C₆)alkyl optionally substituted with —OH, (C₁-C₆)haloalkyl, (C₁-C₆)haloalkoxy, (C₁-C₆)haloalkylthio, —NR³R⁴, —OR³, —C(O)R³, —OC(O)R³, —C(O)NR³R⁴, —C(O)OR³, —SR³, —SO₂R³, and —SO₂NR³R⁴;
   Z₁ and Z₂ are independently selected in each instance from hydrogen, halogen, nitro, cyano, azido, (C₁-C₆)alkyl, (C₁-C₆)haloalkyl, (C₁-C₆)haloalkoxy, (C₁-C₆)haloalkylthio, —NR³R⁴, —NR³C(O)R⁴, —NR³C(O)OR⁵, —OR³, —C(O)R³, —OC(O)R³, —C(O)NR³R⁴, —C(O)OR³, —SR³, —SO₂R³, —SO₂NR³R⁴, and five membered heterocyclyl;
   R^{B} is selected from H, or lower alkyl
   R^{Q} is selected from H, optionally substituted lower alkyl, aryl
   R³, R⁴ and R⁵ are lower alkyl.

In a second aspect, the disclosure relates to pharmaceutical compositions comprising the compounds described herein.

In a third aspect, the disclosure relates to methods and uses of the above-described compounds in medicine, particularly for the treatment of a disease chosen from (a) cancer; (b) diabetes; (c) autoimmune disease; (d) age onset proteotoxic disease (particularly neurodegenerative disease); (e) mood disorder; (f) acne vulgaris; (g) solid organ transplant rejection (graft vs. host disease); (h) pulmonary disease (such as COPD or IPF); (i) cardiac hypertrophy and heart failure; (j) viral or parasitic infection; (k) inflammatory conditions (such as asthma) and (l) organ fibrosis (such as kidney fibrosis). These methods include administering to a patient a therapeutically effective amount of a compound described herein.

In a fourth aspect, the disclosure relates to a method for restoring sensitivity to one or more chemotherapeutic agents in the treatment of cancer. The method includes administering an effective amount of a compound described herein.

In a fifth aspect, the disclosure relates to a method for treating a disease or disorder in a patient where the disease or disorder involves the dysregulation of PP2A influenced signaling cascades such as the PI3K-AKT and MAP kinase pathways. These methods include administering to a patient a therapeutically effective amount of a compound described herein.

In a sixth aspect, the disclosure relates to a method for treating a disease or disorder in a patient where the disease or disorder involves the dysregulation of a Myc dependent signaling pathway. These methods include administering to a patient a therapeutically effective amount of a compound described herein.

In a seventh aspect, the disclosure relates to a method for treating a metabolic disease or disorder in a patient where the disease or disorder involves the dysregulation of the mTOR-PP2A signaling axis. The method includes administering an effective amount of a compound described herein.

In an eighth aspect, the disclosure relates to a method for treating disease or disorder in a patient where the disease or disorder involves cellular hyperproliferation or growth due to dysregulation of the PP2A-Integrator-RNAPII axis.

### DETAILED DESCRIPTION

### Definitions

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or composition that “comprises”, “has”, “includes” or “contains” one or more steps or elements possesses those one or more steps or elements, but is not limited to possessing only those one or more steps or elements. Likewise, a step of a method or an element of a composition that “comprises”, “has” “includes” or “contains” one or more features possesses those one or more features, but is not limited to possessing only those one or more features. The terms “comprising” and “including” or grammatical variants thereof are to be taken as specifying the stated features, integers, steps or components but do not preclude the addition of one or more additional features, integers, steps, components or groups thereof. For example, “X includes a, b and c” means that X includes, but is not limited to, a, b and c. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof, but only if the additional features, integers, steps, components or groups thereof do not materially alter the basic and novel characteristics of the claimed composition or method.

Unless otherwise specified, the phrase “such as” is intended to be open-ended. For example, “X can be a halogen, such as fluorine or chlorine” means that X can be, but is not limited to, fluorine or chlorine.

As used herein, and as would be understood by the person of skill in the art, the recitation of “a compound”—unless expressly further limited—is intended to include salts of that compound. In a particular embodiment, the term “compound of formula” refers to the compound or a pharmaceutically acceptable salt thereof.

The term “pharmaceutically acceptable salt” refers to salts prepared from pharmaceutically acceptable non-toxic acids or bases including inorganic acids and bases and organic acids and bases. When the compounds of the present disclosure are basic, salts may be prepared from pharmaceutically acceptable non-toxic acids including inorganic and organic acids. Suitable pharmaceutically acceptable acid addition salts for the compounds of the present disclosure include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfuric, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present disclosure include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

The terms “subject” or “subject in need thereof” or “patient” are used interchangeably herein. These terms refer to a patient who has been diagnosed with the underlying disorder to be treated. The subject may currently be experiencing symptoms associated with the disorder or may have experienced symptoms in the past. Additionally, a “subject in need thereof” may be a patient at risk of developing a particular disease, or to a patient reporting one or more of the physiological systems of a disease, even though a diagnosis of this disease may not have been made. As a non-limiting example, a “subject in need thereof”, for purposes of this application, may include a male who is currently diagnosed with prostate cancer or was diagnosed with prostate cancer in the past, regardless of current symptomatology.

A “patient,” as used herein, includes both humans and other animals, particularly mammals. Thus the methods are applicable to both human therapy and veterinary applications. In some embodiments, the patient is a mammal, for example, a primate. In some embodiments, the patient is a human.

As used herein, the terms “treatment” or “treating” are used interchangeably. These terms refer to an approach for obtaining beneficial or desired results including, but not limited to, therapeutic benefit. Therapeutic benefit includes eradication or amelioration of the underlying disorder being treated; it also includes the eradication or amelioration of one or more of the symptoms associated with the underlying disorder such that an improvement is observed in the patient, notwithstanding that the patient may still be afflicted with the underlying disorder.

Treatment can involve administering a compound described herein to a patient diagnosed with a disease, and may involve administering the compound to a patient who does not have active symptoms. Conversely, treatment may involve administering the compositions to a patient at risk of developing a particular disease, or to a patient reporting one or more of the physiological symptoms of a disease, even though a diagnosis of this disease may not have been made.

The terms “administer” “administering” or “administration” in reference to a dosage form of the disclosure refers to the act of introducing the dosage form into the system of subject in need of treatment. When a dosage form of the disclosure is given in combination with one or more other active agents (in their respective dosage forms), “administration” and its variants are each understood to include concurrent and/or sequential introduction of the dosage form and the other active agents. Administration of any of the described dosage forms includes parallel administration, co-administration or sequential administration. In some situations, the therapies are administered at approximately the same time, e.g., within about a few seconds to a few hours of one another.

A “therapeutically effective” amount of the compounds described herein is typically one which is sufficient to achieve the desired effect and may vary according to the nature and severity of the disease condition, and the potency of the compound. It will be appreciated that different concentrations may be employed for prophylaxis than for treatment of an active disease. A therapeutic benefit is achieved with the amelioration of one or more of the physiological symptoms associated with the underlying disorder such that an improvement is observed in the patient, notwithstanding that the patient may still be afflicted with the underlying disorder.

The term modulate with respect to PP2A refers to activation or potentiation of phosphatase activity by three general effects. 1. direct allosteric activation of catalytic activity in PP2A complexes. 2. By promotion of assembly of heterotrimeric B-subunit containing trimers, or recruitment of PP2A AC heterodimers to the Integrator-RNAPII complex. 3. By displacement of endogeneous PP2A inhibitors or chaparones, thereby derepressing PP2A activity. These effects are not mutually exclusive though their relative importance may depend on cell or tissue type and specific disease state or pathology.

The term “modulate” with respect to a FOXO transcription factor protein refers to activation of the FOXO transcription factor protein and its biological activities associated with the FOXO pathway. Modulation of FOXO transcription factor proteins includes up-regulation (i.e., agonizing, activation or stimulation). The mode of action of a FOXO modulator can be direct, e.g., through binding to the FOXO transcription factor protein as a ligand. The modulation can also be indirect, e.g., through binding to and/or modifying another molecule which otherwise binds to and activates the FOXO transcription factor protein.

“Hydrocarbon” (e.g., (C₁-C₈)hydrocarbon) includes alkyl, cycloalkyl, polycycloalkyl, alkenyl, alknyl, aryl and combinations thereof. Examples include benzyl, phenethyl, cyclohexylmethyl, adamantyl, norbornyl, and naphthylethyl. Hydrocarbyl (or hydrocarbon) refers to any substituent comprised of hydrogen and carbon as the only elemental constituents. Aliphatic hydrocarbons are hydrocarbons that are not aromatic; they may be saturated or unsaturated, cyclic, linear or branched. Examples of aliphatic hydrocarbons include isopropyl, 2-butenyl, 2-butynyl, cyclopentyl, norbornyl, etc. Aromatic hydrocarbons include benzene (phenyl), naphthalene (naphthyl), anthracene, etc.

Unless otherwise specified, “alkyl” (or alkylene) is intended to include linear or branched saturated hydrocarbon structures and combinations thereof. Alkyl refers to alkyl groups from 1 to 20 carbon atoms, preferably 1 to 10 carbon atoms, more preferably 1 to 6 carbon atoms. Examples of alkyl groups include methyl, ethyl, propyl, isopropyl, n-butyl, s-butyl, t-butyl and the like.

“Cycloalkyl” is a subset of hydrocarbon and includes cyclic hydrocarbon groups of from 3 to 8 carbon atoms. Examples of cycloalkyl groups include cy-propyl, cy-butyl, cy-pentyl, norbornyl and the like.

“Alkoxy” or alkoxyl refers to groups of from 1 to 20 carbon atoms, preferably 1 to 10 carbon atoms, more preferably 1 to 6 carbon atoms of a straight or branched configuration attached to the parent structure through an oxygen. Examples include methoxy, ethoxy, propoxy, isopropoxy and the like. Lower-alkoxy refers to groups containing one to four carbons. For the purpose of this application, alkoxy and lower alkoxy include methylenedioxy and ethylenedioxy.

The term “halogen” means fluorine, chlorine, bromine or iodine atoms. In one embodiment, halogen may be a fluorine or chlorine atom.

The terms “haloalkyl,” “haloalkoxy,” or “haloalkylthio” mean alkyl, alkoxy, or alkylthio, respectively, substituted with one or more halogen atoms.

“Heterocycle” means an aliphatic or aromatic carbocycle residue in which from one to four carbons is replaced by a heteroatom selected from the group consisting of N, O, and S. The nitrogen and sulfur heteroatoms may optionally be oxidized, and the nitrogen heteroatom may optionally be quaternized. Unless otherwise specified, a heterocycle may be non-aromatic (heteroaliphatic) or aromatic (heteroaryl). Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl (also historically called thiophenyl), benzothienyl, thiamorpholinyl, oxadiazolyl, triazolyl and tetrahydroquinolinyl. Examples of heteroaryls include imidazole, pyridine, indole, thiophene, benzopyranone, thiazole, furan, benzimidazole, quinoline, isoquinoline, quinoxaline, pyrimidine, pyrazine, tetrazole and pyrazole. In some embodiments, examples of heteroaryls include imidazole, pyridine, thiophene, thiazole, furan, pyrimidine, pyrazine, tetrazole and pyrazole.

As used herein, the term “optionally substituted” may be used interchangeably with “unsubstituted or substituted”. The term “substituted” refers to the replacement of one or more hydrogen atoms in a specified group with a specified radical. “Oxo” may also be included among the substituents referred to in “optionally substituted”; it will be appreciated by persons of skill in the art that, because oxo is a divalent radical, there are circumstances in which it will not be appropriate as a substituent (e.g. on phenyl). In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine.

Throughout this specification the terms and substituents retain their definitions.

Substituents are generally defined when introduced and retain that definition throughout the specification and in all independent claims.

In one aspect, the disclosure relates to compounds of formula (I):

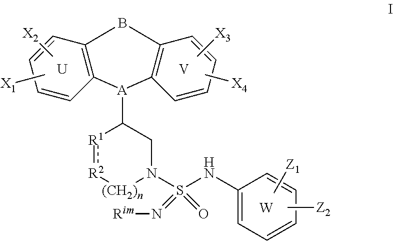

or a pharmaceutically acceptable salt thereof,
as described above. In another aspect, the disclosure relates to compositions comprising compounds of formula (I).

In some embodiments, the disclosure relates to compounds of formula Ia or Ib, or a pharmaceutically acceptable salt thereof, which are stereoisomers at the chiral center on the central ring:

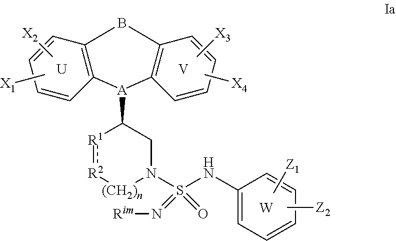

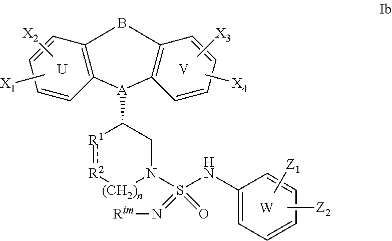

In addition to stereogenic carbon centers on the tetrahydopyran ring the sulfur atom of the sulfonimidamide is also a stereogenic chiral center and the stereoisomers above, Ia and Ib, may occur as diastereoisomers with either configuration at the sulfur center:

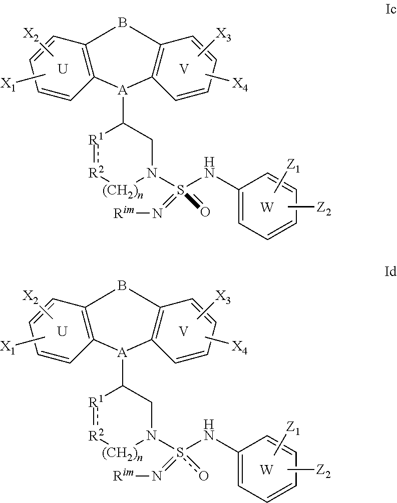

In the embodiments described herein, the compound may be of formula I, Ia, Ib, Ic, or Id, unless otherwise indicated.

In some embodiments A is N. In some embodiments A is CH.

In some embodiments U, V and W are carbocyclic aromatic.

In some embodiments, X₁, X₂, X₃ and X₄ are independently selected in each instance from hydrogen and halogen.

In some embodiments, Z₁ and Z₂ are independently selected in each instance from hydrogen and (C₁-C₆)haloalkoxy.

In some embodiments, B is a direct bond or —CH₂CH₂—.

In some embodiments, R¹ and R² are both CH₂.

In some embodiments, R^{im} is H.

In some embodiments, n is 1.

In some embodiments A is N, U, V and W are carbocyclic aromatic, R¹ is CH₂, R² is CH₂ and n=1, to give a compound of formula II

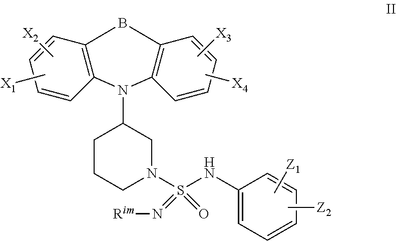

In some embodiments A is CH, U, V and W are carbocyclic aromatic, R¹ is CH₂, R² is CH₂ and n=1, to give a compound of formula III

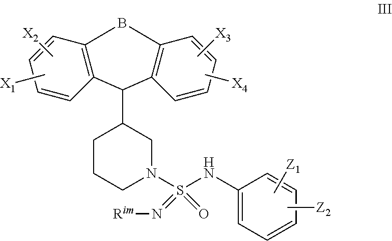

or a pharmaceutically acceptable salt thereof.

In some embodiments, provided is a compound selected from

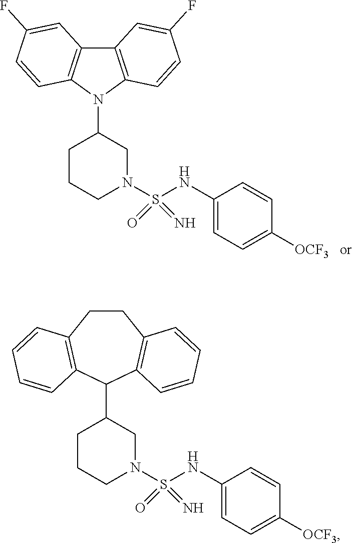

or a pharmaceutically acceptable salt thereof.

The compounds described herein contain asymmetric centers and may thus give rise to enantiomers, diastereomers, and other stereoisomeric forms which may be defined in terms of absolute stereochemistry as (R)- or (S)-. The present disclosure is meant to include all such possible diastereomers as well as their racemic and optically pure forms. Optically active (R)- and (S)-isomers may be prepared using homo-chiral synthons or homo-chiral reagents. Final compounds or intermediates may be resolved in to their enantiomers using conventional techniques such crystallization of amine intermediates with chiral acids or preparative chiral chromatography on final compounds or intermediates. When the compounds described herein contain olefinic double bonds or other centers of geometric asymmetry, and unless specified otherwise, it is intended to include both (E)- and (Z)-geometric isomers. Likewise, all tautomeric forms are intended to be included.

All the members of the genus described above exhibit biological activity in screens that are predictive of utility. However, it may be found upon examination that certain species and genera are not patentable to the inventors in this application. In this case, the exclusion of species and genera in applicants' claims are to be considered artifacts of patent prosecution and not reflective of the inventors' concept or description of their disclosure, which encompasses all members of the genus I that are not in the public's possession.

Also provided herein is a pharmaceutical composition comprising a compound disclosed above, or a pharmaceutically acceptable salt form thereof, and a pharmaceutically acceptable carrier or diluent.

While it may be possible for the compounds of formula I to be administered as the raw chemical, it is preferable to present them as a pharmaceutical composition. According to a further aspect, the present disclosure provides a pharmaceutical composition comprising a compound of formula I or a pharmaceutically acceptable salt thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

Formulations of the compounds and compositions described herein may be administered by a variety of methods: oral (including, but not limited to, capsules, cachets, tablets, powder, granules, solutions, suspensions, emulsions, tablets, or sublingual tablets), buccal, by inhalation (by using, for instance, an inhaler, a nebulizer, an aerosol, a gas, etc.), nasal, topical (including, but not limited to, lotions, creams, ointments, patches (i.e., transdermal), gels, liniments, pastes), ophthalmic, to the ear, rectal (for instance, by using a suppository or an enema), vaginal, or parenteral, depending on the severity and type of the disease being treated. In some embodiments, the compositions are administered orally or intravenously. The formulations include those suitable for oral, parenteral (including subcutaneous, intradermal, intramuscular, intracranial, intravenous and intraarticular), rectal, vaginal, nasal (inhalation), and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula (I) or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

It will be recognized that the compounds of this disclosure can exist in radiolabeled form. i.e., the compounds may contain one or more atoms containing an atomic mass or mass number different from the atomic mass or mass number usually found in nature. Radioisotopes of hydrogen, carbon, phosphorous, fluorine, and chlorine include ²H, ³H, ¹³C, ¹⁴C, ¹⁵N, ³⁵S, ¹⁸F, and ³⁶Cl, respectively. Compounds that contain those radioisotopes and/or other radioisotopes of other atoms are within the scope of this disclosure. Tritiated, i.e. ³H, and carbon-14, i.e., ¹⁴C, radioisotopes are particularly preferred for their ease in preparation and detectability. Compounds that contain isotopes ¹¹C, ¹³N, ¹⁵O and ¹⁸F are well suited for positron emission tomography. Radiolabeled compounds of formula I of this disclosure and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

The compounds provided herein can be used for treating cancer in a patient, the method comprising administering to the patient a therapeutically effective amount of a compound of formula I. In some embodiments, the cancer is characterized by dysregulation of the PI3K-AKT-FOXO signaling pathway. For example, the cancer can be selected from the group consisting of ovarian, pancreatic, renal cell, breast, prostate, lung, hepatocellular carcinoma, glioma, leukemia, lymphoma, colorectal cancers, and sarcomas.

In some embodiments, the cancer is chemotherapy resistant cancer. In some embodiments, the method further comprises administering one or more cancer chemotherapeutic agents. In some embodiments, the one or more cancer chemotherapeutic agents are EGFR inhibitors.

In some embodiments, the cancer is chemotherapy resistant cancer. In some embodiments, the method further comprises administering one or more cancer chemotherapeutic agents targeting transcriptional dysregulation. In some embodiments, the one or more cancer chemotherapeutic agents are CDK inhibitors, particularly CDK9 and/or CDK7 inhibitors.

In some embodiments, the cancer is chemotherapy resistant cancer. In some embodiments, the method further comprises administering one or more cancer chemotherapeutic agents. In some embodiments, the one or more cancer chemotherapeutic agents are mTOR inhibitors.

Certain cancers are characterized by dysregulation and overactivation of cellular transcription carried out by RNA polymerase II (RNAPII) as described in Transcriptional Addiction in Cancer. Bradner, J. E., Hnisz, D. and Young, R. A. February 2017, Cell, Vol. 168, pp. 629-643. BRD4, a member of the bromodomain and extra-terminal domain (BET) family of epigenetic readers, occupies super-enhancers co-opted in transcriptionally addicted cancer cells, and recruits pTEFb to RNAPII to enable promoter proximal pause release and maintain productive elongation (see, for example, Control of Embryonic Stem Cell Identity by BRD4-Dependent Transcriptional Elongation of Super-Enhancer-Associated Pluripotency Genes. Di Micco, R., et al. October 2014, Cell Reports, Vol. 9, pp. 234-247). Therefore BRD4 inhibitor (BRD4i) treatment synergizes with PP2A activation in two ways, first by suppressing BRD4 mediated recruitment of pTEFb to paused RNAPII. A second mode of synergy with BRD4i treatment is by reversing the activating phosphorylation of BRD4 itself by PP2A complexes (see Phospho-BRD4: transcription plasticity and drug targeting. Chiang, C. 2016, Drug Discov Today: Technol, http://dx.doi.org/10.1016). The compounds provided herein can be used for treating cancer in a patient where there is transcriptional dysregulation, the method comprising administering to the patient a therapeutically effective amount of a compound of formula I and a BRD4 inhibitor. Cancers characterized by transcriptional addiction include Breast Cancer, Osteosarcoma, Endometrial Cancer, Acute Myeloid Leukemia, Lung Cancer, Prostate Cancer, Melanoma and Ovarian Cancer.

In some embodiments, administration of a compound of formula I can restore sensitivity to one or more chemotherapeutic agents in a patient wherein the patient has developed a resistance to the one or more chemotherapeutic agents. More particularly, cancers that may be treated by the compounds, compositions and methods described herein include, but are not limited to, the following:
cardiac cancers, including, for example sarcoma, e.g., angiosarcoma, fibrosarcoma, rhabdomyosarcoma, and liposarcoma; myxoma; rhabdomyoma; fibroma; lipoma and teratoma;
lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma;
gastrointestinal cancer, including, for example, cancers of the esophagus, e.g., squamous cell carcinoma, adenocarcinoma, leiomyosarcoma, and lymphoma: cancers of the stomach. e.g., carcinoma, lymphoma, and leiomyosarcoma: cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma:
genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma;
liver cancers, including, for example, hepatoma, e.g., hepatocellular carcinoma; cholangiocarcinoma: hepatoblastoma; angiosarcoma; hepatocellular adenoma; and hemangioma;
bone cancers, including, for example, osteogenic sarcoma (osteosarcoma), fibrosarcoma, malignant fibrous histiocytoma, chondrosarcoma, Ewing's sarcoma,
malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors;
nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma;
gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma: cancers of the vagina, e.g., clear cell carcinoma, squamous cell carcinoma, botryoid sarcoma, and embryonal rhabdomyosarcoma; and cancers of the fallopian tubes, e.g., carcinoma;
hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenstrom's macroglobulinemia;
skin cancers, including, for example, malignant melanoma, basal cell carcinoma, squamous cell carcinoma, Kaposi's sarcoma, moles dysplastic nevi, lipoma, angioma, dermatofibroma, keloids, psoriasis; and adrenal gland cancers, including, for example, neuroblastoma.

Cancers may be solid tumors that may or may not be metastatic. Cancers may also occur, as in leukemia, as a diffuse tissue.

The compounds described herein can also be administered in combination with existing methods of treating cancers, for example by chemotherapy, irradiation, or surgery. Thus, there is further provided a method of treating cancer comprising administering an effective amount of a compound according to formula I to a patient, wherein a therapeutically effective amount of one or more additional cancer chemotherapeutic agents are administered to the patient.

Also provided herein is a method for treating diabetes in a patient, the method comprising administering to the patient a therapeutically effective amount of a compound of formula I.

Further provided herein is a method for treating an autoimmune disease in a patient, the method comprising administering to the patient a therapeutically effective amount of a compound of formula I. The autoimmune disease can be, for example, inflammatory bowel disease (IBD). Immune responses are constantly and tightly regulated and one important cellular component in maintaining self tolerance (i.e., prevention of autoimmunity) and tolerance of benign commensal gut flora are regulatory T cells (Treg). Treg can be subdivided into multiple phenotypes, but the most common are CD4+CD25+ T cells that express the transcription factor Foxp3. Foxp3 is a direct transcriptional target of FOXO proteins, particularly FOXO1 and FOXO3. Thus activation of FOXO proteins in naïve T-cells promotes and directs differentiation to maintain a population of Treg cells.

Acute immune mediated rejection and chronic immune mediated rejection are key obstacles to successful solid organ transplantation. It is believed that these forms of rejection can be prevented/overcome by amplifying Treg number and or function. Similarly, a common and morbid complication of allogeneic hematopoietic cell transplants (Allo-HCT) used to treat various malignant and non-malignant conditions, is graft versus host disease, in which the transplanted immune cells from the donor damage multiple organs in the recipient (most notably skin, gut, and liver). Increasing experimental and clinical data indicate that Tregs can be harnessed to prevent and or treat this disease process.

Thus compounds of the present disclosure are useful in treatment of autoimmune and related diseases, by activating FOXO proteins and inducing T cell differentiation to Tregs. Compounds may be administered therapeutically to subjects directly, or alternatively, T cells may be collected from a subject and differentiated ex vivo to Tregs as described by Taylor et al. [Blood 99, 3493-3499 (2002)].

Aspects of the disclosure include methods for treatment of autoimmune disease characterized by deficiency in Treg function comprising administering a therapeutically useful amount of compound of formula I. The method can also include extraction of naïve T-cells from a patient, differentiation of T-cells to Tregs ex vivo by treatment with a compound of formula I, optionally supplemented with an HDACi, followed by administration of Tregs to patient with optional separation of compound of formula I from Tregs prior to their administration. As stated above, autoimmune diseases that can be so treated include IBD, solid organ transplant rejection, and GvHD in allo-HCT.

In some embodiments, the compounds can be administered to a patient to treat an autoimmune disorder, for example, Addison's disease, Amyotrophic Lateral Sclerosis, celiac disease, Crohn's disease, diabetes, eosinophilic fasciitis, Guillain-Barre syndrome (GBS), Graves' disease, Lupus erythematosus, Miller-Fisher syndrome, psoriasis, rheumatoid arthritis, ulcerative colitis, and vasculitis. In some embodiments, the compound provided herein can be used for treating a disease or disorder in a patient wherein the disease or disorder involves excessive or unregulated cellular proliferation, the method comprising administering to the patient a therapeutically effective amount of a compound of formula I. Also provided herein is a method for treating a disease or disorder in a patient where the disease or disorder involves the dysregulation of the PI3K-AKT-FOXO signaling pathway, the method comprising administering to the patient a therapeutically effective amount of a compound of formula I.

Further provided herein is a method for treating a disease in a patient wherein the disease is characterized by proteotoxicity, including age onset proteotoxicity leading to neurodegeneration, the method comprising administering to the patient a therapeutically effective amount of a compound of formula I. Hyperphosphorylated Tau has been implicated as the pathogenic protein in several neurodegenerative diseases and furthermore PP2A has been shown to be an important phosphatase in reversing aberrant phosphorylation of Tau; see for example Ludovic Martin et al., Tau protein phosphatases in Alzheimer's disease: The leading role of PP2A in Ageing Research Reviews 12 (2013) 39-49; Miguel Medina and Jesus Avila, Further understanding of tau phosphorylation: implications for therapy in Expert Rev. Neurotherapy, 15(1), 115-112 (2015) and Michael Voronkov et al., Phosphoprotein phosphatase 2A: a novel druggable target for Alzheimer's disease in Future Med Chem. 2011 May, 3(7) 821-833. Hyperphosphorylated alpha-Synuclein is a second exemplar of a toxic protein, and again PP2A has been shown to reverse its aberrantly phosphorylated state; see for example Kang-Woo Lee et al., Enhanced Phosphatase Activity Attenuates alpha-Synucleinopathy in a Mouse Model in Neurobiology of Disease, May 11, 2011, 31(19) 6963-6971. In some embodiments, the disease is selected from the group consisting of: Alzheimer's disease, Parkinson's disease, Huntington's disease, amyotrophic lateral sclerosis, frontotemporal dementia, progressive supranuclear palsy, corticobasal degeneration and Pick's disease.

A second feature of Alzheimer's disease is deposition of amyloid plaques and phosphorylation of Amyloid Precursor Protein (APP) at threonine-668 in the cytoplasmic domain of APP is involved in it's processing to generate toxic amyloid-beta (see T. Zhang et al, Int. J. Mol. Sci. 2020, 21, 209). Activation of PP2A by treatment with compounds of the present disclosure decreases threonine-668 phosphorylation and suppresses pathological amyloidogenesis contributing to the development of Alzheimer's disease.

The compounds provided herein may further be used in a method for treating a mood disorder in a patient by administering to the patient a therapeutically effective amount of a compound of formula I. In some embodiments, the mood disorder is stress-induced depression.

Also provided herein is a method for treating acne vulgaris in a patient by administering to the patient a therapeutically effective amount of a compound of formula I.

Further provided herein is a method for treating pulmonary disease such as COPD. Protein phosphatase 2A (PP2A) is a primary serine-threonine phosphatase that modulates inflammatory responses in asthma and COPD. PP2A has shown to be dysregulated in mouse models of COPD, and inhibiting PP2A activity exacerbated inflammatory responses in the lung. Conversely, increasing PP2A activity via PP2A protein transfection down regulated cytokine expression and prevented the induction of proteases following cigarette smoke extract (CSE) treatment. Thus, increasing PP2A activity by treatment with compounds of the present disclosure may ameliorate or reverse the pathology underlying lung diseases such as COPD.

Idiopathic Pulmonary Fibrosis is a fatal lung disease in which there is progressive and irreversible scaring of the lung associated with changes to alveolar epithelial cells and aberrant fibroblast proliferation and activation. The underlying causative agent in IPF is usually unknown (hence idiopathic) and the prognosis after diagnosis is dismal with a median survival time of three years. IPF is characterized by a continuous expansion of the fibroblast population and excessive deposition of collagen in the alveolar wall leading to scarred, non-functional airspaces progressive hypoxia and death by asphyxiation. In normal lung tissue fibroblasts interact with the extracellular matix(ECM) and signaling via integrins activates PP2A and this suppresses fibroblast growth and proliferation. In IPF fibroblasts this signaling is defective and PP2A activation is muted; in these aberrant cells, uncontrolled fibroblast proliferation and collagen secretion occurs. Diminished PP2A signaling in PP2A fibroblasts has several consequences: 1. Excessive phosphorylation of the transcription factor FOXO3a, which leads to exit of phospho-FOXO3a from the nucleus to the cytoplasm where it is sequestered by 14-3-3 proteins. PP2A is known to be the phosphatase responsible for dephosphorylating cytoplasmic FOXO3a and promoting it's nuclear translocation. Activated, phospho-Akt is a major kinase responsible for phosphorylation of FOXO3a and PP2A is the phosphatase responsible for dephosphorylating and deactivating Akt. Thus PP2A activation promotes FOXO3a activity in two ways, by suppressing the activity of a major kinase. Akt, that inactivates it, and second by dephosphorylating cytoplasmic phospho-FOXO3a directly to cause nuclear translocation. Deficient nuclear FOXO3 protects IPF fibroblasts from polymerized collagen matrix induced apoptosis, therefore PP2A activation will suppress growth of, and will induce apoptosis of IPF fibroblasts. 2. Low PP2A activity in IPF fibroblasts results in HDAC4 hyperphosphorylation and decreases it's nuclear localization, thus the histones of it's target genes remain acetylated and transcriptionally active which drives excessive collagen secretion from IPF fibroblasts. Thus PP2A activation, by promoting HDAC4 nuclear translocation, will suppress excessive the excessive collagen secretion characteristic of IPF and other systemic fibrotic diseases. 3. Activated phosph-ERK is a direct target of PP2A, which it dephosphorylates and deactivates. In scleroderma fibroblasts TGFb reduces PP2A activity and promotes ERK signaling and excessive collagen production. Activation of PP2A will suppress this signaling pathway from TGFb, a known and important pro-fibrotic cytokine. It is reasonable to conjecture that a similar pathway is operative in lung fibroblasts in IPF, thus PP2A activation should be useful there also. 4. PP2A negatively regulates Wnt/b-catenin signaling. Wnt3a induces lung epithelial cell proliferation, fibroblast activation and collagen synthesis in IPF. PP2A activation will suppress these processes and thus exert a therapeutic benefit in lung fibrosis and IPF. 5. Promotion of RNAPII pausing in hyperactivated lung fibroblasts in IPF by PP2A activation suppresses the expression of fibrosis related genes such as smooth muscle actin (ACTA2), collagen genes (COL1A1, COL1A2 and COL3A1) and fibronectin (FN1) (see Sattar et al, Chemical Activation of Protein Phosphatase 2A Counters TGFβ-Dependent Induction of Extracellular Matrix Proteins in Fibroblasts. Am J Respir Crit Care Med 2022; 205: A1941, poster presented PULMONARY FIBROSIS: ANIMAL AND CELL CULTURE MODELS/Thematic Poster Session/Sunday, May 15, 2022, San Francisco ATS meeting). Therefore compounds of the present disclosure exert a useful therapeutic effect in IPF by suppression of these genes and reducing excesive collagen deposition and scarring in IPF. In summary, PP2A is involved in several major signaling pathways implicated in the pathogenesis of lung fibrosis and IPF and in all the cases cited above PP2A activation is likely to exert a beneficial therapeutic effect. This implies that a well tolerated, effective, small molecule PP2A activator would constitute a novel therapeutic for lung fibrosis.

Impaired PP2A/AKT signaling has been observed in cellular models of idiopathic pulmonary hypertension, where it causes obstructive hyperproliferation and apoptosis resistance of distal pulmonary artery smooth muscle cells. Increasing PP2A activity may reverse this, thus, treatment with compounds of the present disclosure may be an effective treatment for pulmonary hypertension.

Further provided herein is a method for treating cardiac hypertrophy in a patient by administering to the patient a therapeutically effective amount of a compound of formula I. In some embodiments, the cardiac hypertrophy is associated with a disease selected from hypertension, myocardial infarction, heart failure, and valvular heart disease. Cardiac physiology and hypertrophy are regulated by the phosphorylation status of many proteins, including receptors and ion channels, which is partly controlled by a PP2A-alpha4 intracellular signalling axis. Studies indicate that the type 2A protein phosphatases are differentially regulated in both the healthy and hypertrophied myocardium. The data suggest that pressure overload-induced hypertrophy is associated with (1) altered expression of type 2A protein phosphatases and their regulatory subunits and (2) an increase in expression of their non-catalytic inhibitor protein alpha4. Thus, treatment with compounds of the present disclosure may ameliorate cardiac hypertrophy. Also, significant reduction in endosomal PP2A activity has been observed in heart failure samples versus controls, suggesting that inhibited resensitization of beta-adrenergic receptors occurs in human heart failure. These studies suggest that resensitization of beta adrenergic receptors is inhibited in human heart failure and targeting the PP2A inhibitor SET to derepress and activate PP2A may provide preservation of receptor function and beneficial cardiac remodeling. Thus, treatment with compounds of the present disclosure may have a beneficial effect in heart failure.

Further provided herein is a method for treating a parasitic infection in a patient by administering to the patient a therapeutically effective amount of a compound of formula I. Examples of parasites that may cause parasitic infections to be treated include, but are not limited to, Plasmodium and Theileria.

Further provided herein is a method for treating inflammatory conditions. Reduced PP2A activity occurs in animal models of allergic airway disease and patients with severe asthma. Treatment with small molecule activators of PP2A such as fingolimod (FTY720) or 2-amino-4-(4-(heptyloxy) phenyl)-2-methylbutan-1-ol (AAL(S)) inhibited the development of inflammation, airway hyperreactivity in mouse models of allergic airway disease. Thus, compounds of the present disclosure may be useful in the treatment of asthma. Dephosphorylation of tristetraprolin (TTP) functions as an “off-switch” in inflammatory responses, and it's activity can be promoted by compounds that stimulate PP2A activity. Therapeutic efficacy of protein phosphatase 2A (PP2A)-activating drugs, to target tristetraprolin (TTP), in models of rheumatoid arthritis has been demonstrated in vitro and in vivo. Thus, treatment with compounds of the present disclosure may be useful in chronic inflammatory conditions such as rheumatoid arthritis.

PP2A enzymes are involved in the regulation of cell transcription, cell cycle, and viral transformation. Many viruses, including cytomegalovirus, parainfluenza, DNA tumor viruses, and HIV-1, utilize different approaches to exploit PPA2 in order to modify, control, or inactivate cellular activities of the host [Garcia et al., Microbes and Infection, 2, 2000, 401-407]. Therefore, the compounds provided herein may further be used in a method for treating a viral infection in a patient by administering to the patient a therapeutically effective amount of a compound of formula I. Examples of viruses that may cause viral infections to be treated include, but are not limited to: a polyomavirus, such as John Cunningham Virus (JCV), Simian virus 40 (SV40), or BK Virus (BKV); influenza. Human Immunodeficiency Virus type 1 (HIV-1), Human Papilloma Virus (HPV), adenovirus, Epstein-Barr Virus (EBV), Hepatitis C Virus (HCV), Molluscum contagiosum virus (MCV); Human T-lymphotropic virus type 1 HTLV-1), Herpes Simplex Virus type 1 (HSV-1), cytomegalovirus (CMV), hepatitis B virus, Bovine papillomavirus (BPV-1), human T-cell lymphotropic virus type 1, Japanese encephalitis virus, respiratory syncytial virus (RSV), and West Nile virus.

Serine/Threonine phosphatases, including PP2A, are involved in modulation of synaptic plasticity (D. G. Winder and J. D. Sweatt, Nature Reviews Neuroscience, vol 2, July 2001, pages 461-474). Persistently decreased PP2A activity is associated with maintenance of Long Term Potentiation (LTP) of synapses, thus treatment PP2A activators such as those described here may reverse synaptic LTP. Psychostimulant drugs of abuse such as cocaine and methamphetamine are associated with deleterious synaptic LTP (L. Mao et al, Neuron 67, Sep. 9, 2010 and A. Stipanovich et al, Nature vol 453, 2008, pages 879-884), which may underlie the pathology of addiction and relapse therefore PP2A activators described here may be useful as treatments for psychostimulant abuse.

Abnormalities in synaptic structure and signaling are linked to autistic spectrum disorder, see for example, Y Chen et al., CTTNBP2, but not CTTNBP2NL, regulates dendritic spinogenesis and synaptic distribution of the striatin-PP2A complex, Molecular Biology of the Cell, 23, Nov. 15, 2012, 4383-4392. PP2A has been shown to be important in normal development of dendritic spines, and treatment with compounds of the present disclosure may ameliorate or reverse autistic spectrum disorder.

Provided herein is a method of treating a disease related to deficient Protein Phosphatase 2A (PP2A) activity in a subject, comprising administering a compound described herein to a subject in need thereof. In some embodiments, the disease is a cancer, an inflammatory disease, an autoimmune disease, a fibrotic disease, or a neurodegenerative disease.

In some embodiments of the method, the cancer is resistant to at least one previous therapy that was administered to the subject, and administration of a compound described herein resensitizes the cancer to said previous therapy.

In some embodiments, the disease is prostate cancer, breast cancer, lung fibrosis, or Alzheimer's disease.

Further provided herein is a method for treating a disease or disorder in which the disease or disorder involves the dysregulation of the mTOR-PP2A signaling axis. Mammalian target of rapamycin (mTOR) is a serine/threonineprotein kinase that regulates cell growth, proliferation, and survival: mTOR is frequently activated in human cancers and is a commonly sought anticancer therapeutic target. PP2A is a key element in mTOR-AKT signaling during nutritional deprivation, and it has important implications in cell cycle progression and quiescence. Dysregulation of cellular metabolism is a feature of cancer, with nutrient transport defects, nutrient sensing defects, dysregulated autophagy and constitutive anabolism being common in tumors; aberrant activation of mTOR is implicated in all of these processes and PP2A activation has been demonstrated to modulate them in vivo. PP2A has been shown to be involved in regulatory feedback loops with mTOR, and PP2A activators of the present disclosure would be expected to affect these processes directly by interacting with mTOR complexes, or indirectly by counterbalancing mTOR's effects by dephosphorylating its targets. Perturbation of the mTOR signaling cascade appears to be a common pathophysiological feature of human neurological disorders, including mental retardation syndromes and autism spectrum disorders, and neurodegenerative conditions such as Alzhiemer's disease. Activation of PP2A has been shown to be effective in animal models of neurodegenerative disease by modulating the PP2A mTOR axis; thus, molecules of the present disclosure will be useful in treatment of these conditions. PP2A activators of the present disclosure are likely to be useful in the treatment of diseases in which mTOR signaling is dysregulated; these include cancer, diabetes and neurodegenerative conditions. Compounds of the present disclosure may also promote innate immunity to infection and promote healthy aging.

### Abbreviations

Unless defined otherwise, all technical and scientific terms used herein have the same meaning as is commonly understood by one of ordinary skill in the art to which this disclosure belongs. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference. In the event that there is a plurality of definitions for terms cited herein, those in this section prevail unless otherwise stated.

The following abbreviations and terms have the indicated meanings throughout:

| | |
|---|---|
| Ac= | acetyl |
| Ag= | aqueous |
| Boc= | t-butyloxy carbonyl |
| Bu= | butyl |
| c-= | cyclo |
| cat= | catalyst |
| Cbz= | carboxybenzyl |
| DBA= | dibenzylideneacetone |
| DCM= | dichloromethane = methylene chloride = CH₂Cl₂ |
| DMF= | N,N-dimethylformamide |
| eq. or equiv.= | equivalent(s) |
| Et= | ethyl |
| GC= | gas chromatography |
| h= | hour(s) |
| KHMDS= | Potassium bis(trimethylsilyl)amide |
| Lg= | leaving group |
| Ln= | chiral ligands |
| mCPBA= | meta-Chloroperoxybenzoic acid |
| Me= | methyl |
| mesyl= | methanesulfonyl |
| min.= | minute(s) |
| Ms= | mesylate |
| NMO or NMMO= | N-methylmorpholine oxide |
| Pg= | protecting group |
| Ph= | phenyl |
| RT= | room temperature |
| sat'd or sat.= | saturated |
| t- or tert= | tertiary |
| Tf= | triflate |
| TFA= | trifluoroacetic acid |
| THF= | tetrahydrofuran |
| tosyl= | p-toluenesulfonyl |

| Example Number | Structure |
|---|---|
| 1 | 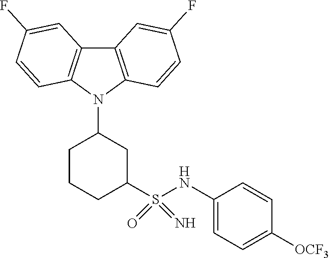 [SMILES: N=S(=O)(Nc1ccc(OC(F)(F)F)cc1)C1CCCC(n2c3ccc(F)cc3c3cc(F)ccc32)C1] |
| 2 | 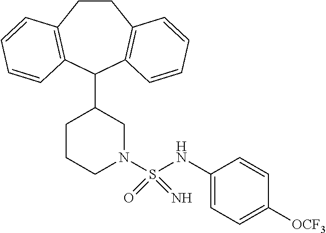 [SMILES: N=S(=O)(Nc1ccc(OC(F)(F)F)cc1)N1CCCC(C2c3ccccc3CCc3ccccc32)C1] |

Preparation of compounds can involve the protection and deprotection of various chemical groups. The need for protection and deprotection, and the selection of appropriate protecting groups, can be readily determined by one skilled in the art. Suitable groups for that purpose are discussed in standard textbooks in the field of chemistry, such as Protective Groups in Organic Synthesis by T. W. Greene and P. G. M. Wuts [John Wiley & Sons, New York, 1999], in Protecting Group Chemistry, 1^{st} Ed., Oxford University Press, 2000; and in March's Advanced Organic chemistry: Reactions, Mechanisms, and Structure, 5^{th} Ed., Wiley-Interscience Publication, 2001.

Sulfonimidamides are isosteres of sulfonamides and their application in medicinal chemistry has been reviewed in Chinthakindi et al, Angew. Chem. Int. Ed. 2017, 56, 4100-4109 and Nandi and Arvidsson, Sulfonimidamides: Synthesis and Applications in Preparative Organic Chemistry, Adv. Synth. Catal., 2018, 360, 2976-3001 Sulfonimidamides may improve physical and medicinal properties, versus the corresponding sulfonamides, with respect to solubility, lipophilicity, plasma protein binding or permeability. They may also have altered, and potentially beneficial, off-target profile versus the sulfonamides with respect to, for example CYP inhibition or PXR induction resulting in reduced potential for drug-drug inteactions or toxicity.

Compounds of formula I, II and III of the present disclosure are immide analogs of a sulfonyl urea. They are prepared from the sulfonyl ureas using conditions shown in General Synthesis Scheme 1. The sulfonyl urea starting materials are prepared by the methods disclosed in PCT/US2021/023234 and PCT/US2021/014342.

Conditions in General Synthesis Scheme 1 are adapted from those published in Chen and Gibson, RSC Adv. 2015, 5, 4171-4174 or Chinthakindi et al. Eur. J. Org. Chem., 2018, 10.1002/ejoc.201801541. In summary the requisite sulfonyl urea staring material dissolved in dry aprotic solvent such as chloroform and a tertiary amine base such as triethylamine or Hunigs base (1-2 equivalents) is added. The mixture is treated with Ph₃PCl₂ (1-2 equivalent) with optional conventional heating or under microwave conditions. The mixture is cooled to room temperature and a primary amine R^{im}NH2 is added and the mixture is stirred for 1-24 hrs. Standard aqueous work-up and purification gives the product. The R^{im} moiety may optionally be a protecting group, which is removed to give compounds wherein R^{im} is hydrogen.

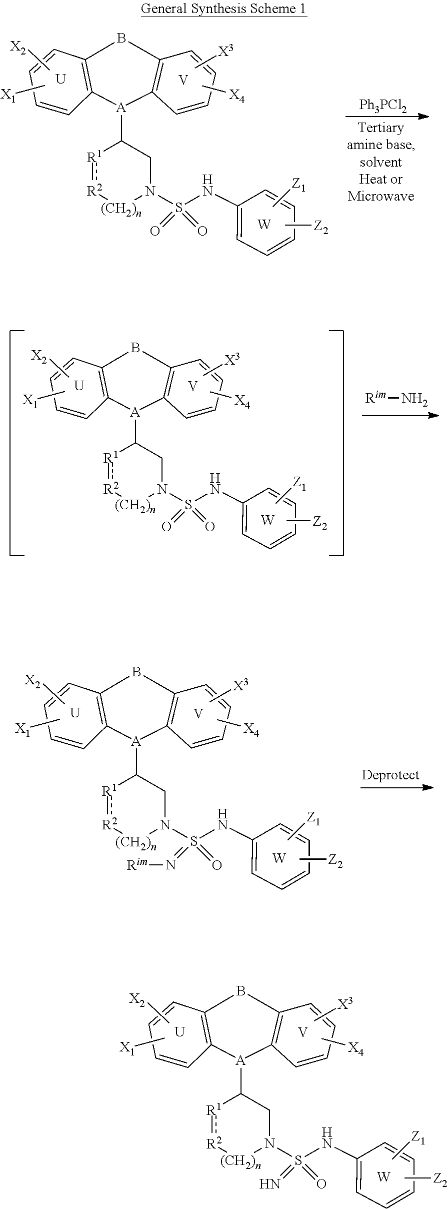

Synthesis of Example 1: Synthetic route to 3-(3,6-difluoro-9H-carbazol-9-yl)-N-(4-(trifluoromethoxy)phenyl)piperidine-1-sulfonimidamide is shown in Scheme 2 below:

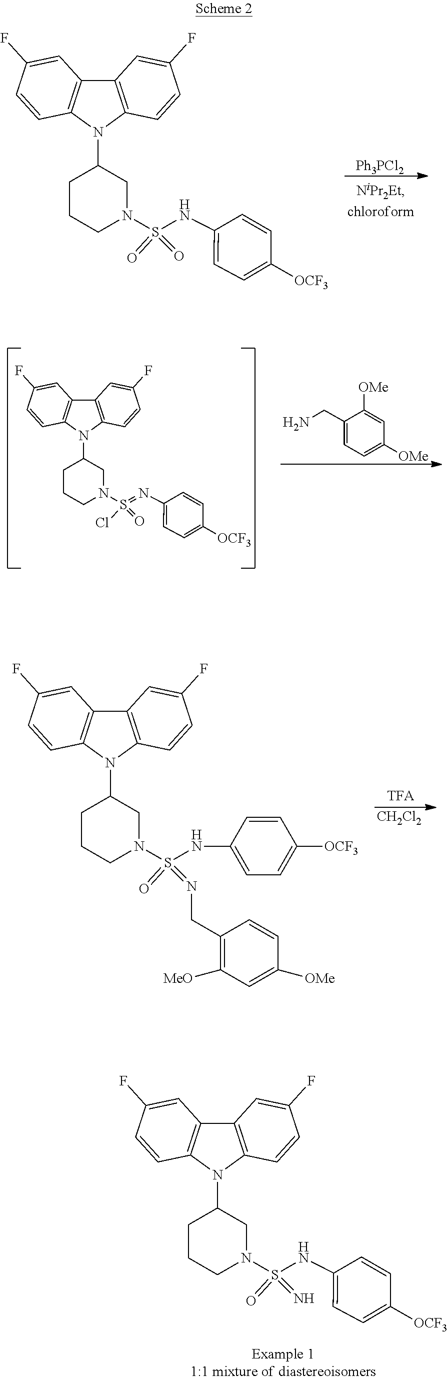

3-(3,6-difluoro-9H-carbazol-9-yl)-N-(4-(trifluoromethoxy)phenyl)piperidine-1-sulfonamide was prepared by the methods disclosed in PCT/US2021/023234 and PCT/US2021/014342.

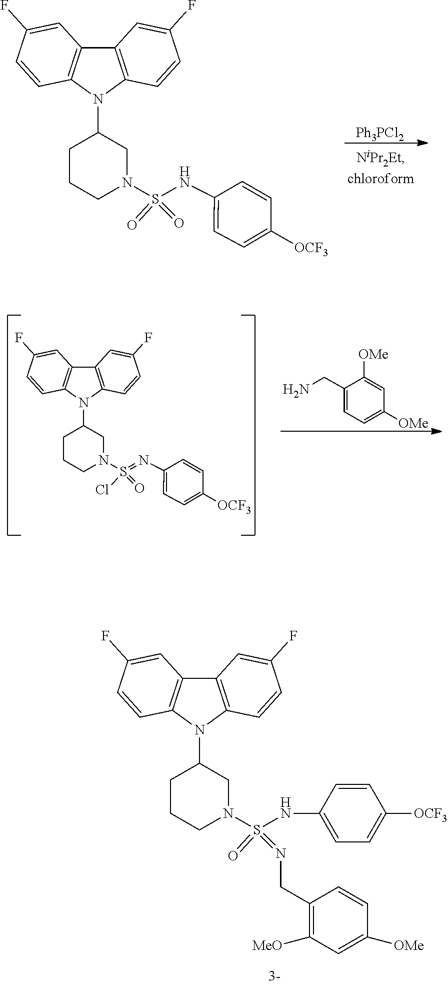

Step 1: (3,6-difluoro-9H-carbazol-9-yl)-N-(4-(trifluoromethoxy)phenyl)piperidine-1-sulfonamide, 0.4 g (0.76 mmole, 1 equiv) was dissolved in 10 mL dry chloroform in a microwave reaction vessel. Hunigs base 0.34 mL (0.26 g, 2 mmole,) was added, followed by 0.5 g (1.5 mmole, 2 equiv) of dichlorotriphenyl-λ⁵-phosphane as a solid in one portion. The vessel was capped and the mixture was stirred and microwaved at 85° C. for 20 minutes (dark brown-black color develops). The mixture was cooled to room temperature then 0.67 g (4 mmole) of (2,4-dimethoxyphenyl)methanamine was added and the mixture was stirred overnight. The reaction was diluted into 100 mL ethyl acetate and washed with 100 mL water. The organic layer dried over anhydrous sodium sulfate. Filtration and evaporation gave crude material which was purified by flash chromatography, eluting with 20% ethyl acetate/hexane. The product, 3-(3,6-difluoro-9H-carbazol-9-yl)-N-(2,4-dimethoxybenzyl)-N-(4-(trifluoromethoxy)phenyl)piperidine-1-sulfonimidamide, was obtained as an oil which formed foam on pumping in vacuo, 0.12 g, 0.18 mmole, 23%. 400 MHz ¹H NMR in CDCl₃ is consistent with a 1:1 mixture of diastereoisomers (sulfur atom is a chiral center). MS ESI −ve ion 673.0 [M−H]⁻ and 523.9 (fragment after loss of PMB).

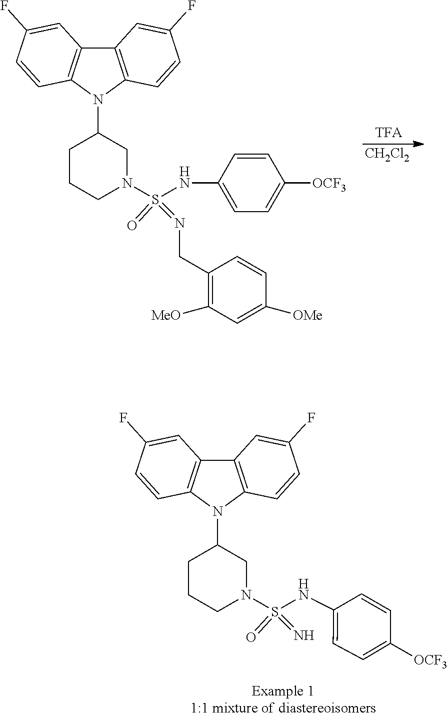

Step 2: The PMB protected compound, 0.1 g was dissolved in 10 mL methylene chloride then 10 mL trifluoroacetic acid was added and solution turned from pale yellow to deep purple on stirring at room temperature overnight. The reaction was evaporated and residue was portioned between 50 mL ethyl actetate and 50 mL sat. aq. sodium bicarbonate. The organic was dried over anhydrous sodium sulfate, the filtered and evaporated to give the crude product as a oil that was purified by flash chromatography. 400 MHz ¹H NMR in CDCl₃ is consistent with a 1:1 mixture of diastereoisomers (sulfur atom is a chiral center). MS ESI −ve 522.9 [M−H]; +ve 524.9 [M+H]⁺ and 546.9 [M+Na]′⁻.

Synthesis of Example 2: Synthetic route to 3-(3,6-difluoro-9H-carbazol-9-yl)-N-(4-(trifluoromethoxy)phenyl)piperidine-1-sulfonimidamide is shown in Scheme 3 below:

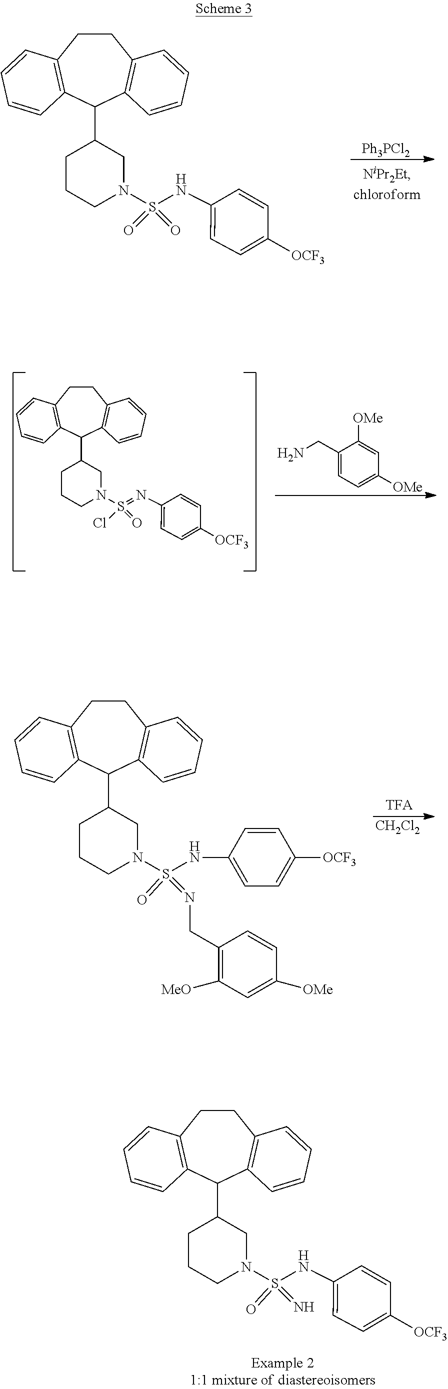

3-(10,11-dihydro-5H-dibenzo[a,d][7]annulen-5-yl)-N-(4-(trifluoromethoxy)phenyl)piperidine-1-sulfonamide was prepared by the methods disclosed in PCT/US2021/023234.

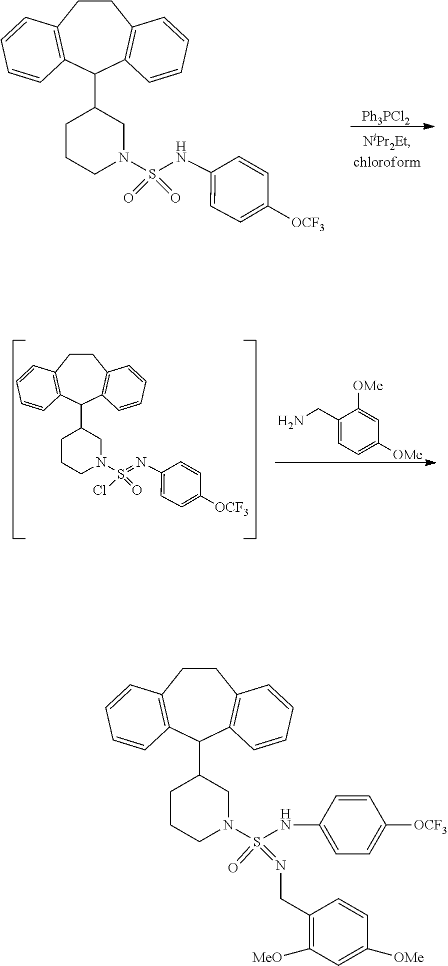

Step 1: 3-(10,11-dihydro-5H-dibenzo[a,d][7]annulen-5-yl)-N-(4-(trifluoromethoxy)phenyl)piperidine-1-sulfonamide, 0.522 g (1 mmole, 1 equiv) was dissolved in 10 mL dry chloroform in a microwave reaction vessel. Hunigs base 0.34 mL (0.26 g, 2 mmole, 2 equiv) was added, followed by 0.5 g (1.5 mmole. 1.5 equiv) of dichlorotriphenyl-λ⁵-phosphane as a solid in one portion. The vessel was capped and the mixture was stirred and microwaved at 85° C. for 20 minutes (dark brown-black color develops). The mixture was cooled to room temperature then 0.67 g (4 mmole, 4 equiv) of (2,4-dimethoxyphenyl)methanamine was added and the mixture was stirred overnight. A thick white precipitate forms overnight. The reaction was partitioned between 100 mL ethyl acetate and 100 mL 0.1 M aqueous hydrochloric acid. The organic layer was washed once with saturated aqueous sodium bicarbonate the dried over anhydrous sodium sulfate. Filtration and evaporation gave crude material which was purified by flash chromatography, eluting with 20% ethyl acetate/hexane. The product, 3-(10.11-dihydro-5H-dibenzo[a,d][7]annulen-5-yl)-N-(2,4-dimethoxybenzyl)-N-(4-(trifluoromethoxy)phenyl) piperidine-1-sulfonimidamide, was obtained as an oil which formed foam on pumping in vacuo, 0.45 g, 68%. 400 MHz ¹H NMR in CDCl₃ is consistent with a 1:1 mixture of diastereoisomers (sulfur atom is a chiral center). TLC-MS APCI +ve 666.9 [M+H]⁺, −ve 664.8 [M−H]⁻.

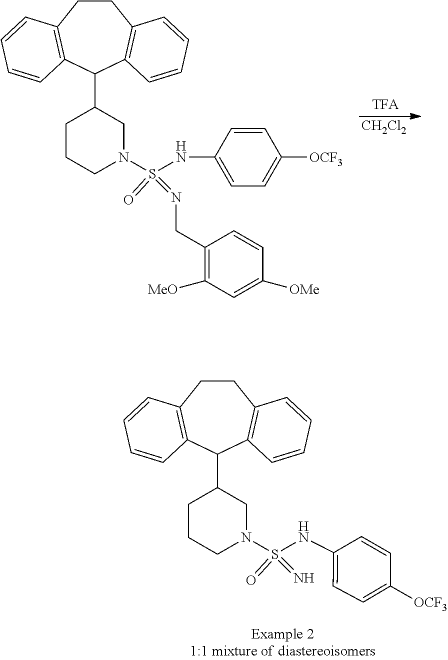

The PMB protected intermediate, 0.45 (0.68 mmole) was dissolved in 5 mL methylene chloride then 5 mL trifluoroacetic acid was added and solution turned from pale yellow to deep purple on stirring at room temperature overnight. The reaction was quenched by pouring onto 10 g potassium carbonate in 100 mL water and 100 mL ethyl acetate. The organic was dried over anhydrous sodium sulfate, the filtered and evaporated to give the crude product as a oil that was purified by flash chromatography, eluting with 305 ethyl acetate/hexane. 400 MHz ¹H NMR in CDCl₃ is consistent with a 1:1 mixture of diastereoisomers (sulfur atom is a chiral center). TLC-MS APCI +ve 515.5 [M+H]⁺, −ve 513.7 [M−H]⁻.

### Cancer Cell Viability Inhibition

D425 medulloblastoma cells were purchased from SigmaAldrich. D425 meduloblastoma cells were cultured in improved Dulbecco's modified Eagle's medium (DMEM) Richter's modification supplemented with 20% fetal bovine serum and 1% Penicillin-streptomycin. Incubation was performed at 37° C. and 5% CO₂.

D425 cells were seeded at 3000-3500 cells/90 ul growth medium per well on 96-well tissue culture plates. Cells were incubated overnight to allow them to recover. Next day cells were treated with tested compounds and incubated for 48 hours.

Cell Proliferation assays in triplicate were performed at each concentration. Compounds test range was 1-30 uM (0, 5, 10, 15, 20, 25, and 30). The compounds were dissolved in DMSO. A series of dilutions were made in 1% DMSO in growth medium so that the final concentration of DMSO is 0.1% in all of treatments.

After treatment, cells were allowed to equilibrate at room temperature for one hour. Cell proliferation was measured by Luminescence quantification using Promega CellTiter-Glo Luminescent Cell Viability Assay. To perform the assay, 90 ul of Celltiter-Glo substrate was added to each well, plates were shake for 2 minutes and allowed to equilibrate for 10 minutes at room temperature. Luminescence intensity was measured using the Spectramax i3× plate reader.

The Luminescence intensity data were analyzed using the computer software Graphpad Prism. In the absence of the compound, the luminescence intensity (Lₜ) in each data set was defined as 100% cell viability. The percent cell in the presence of each compound was calculated according to the following equation: % Cell=L/Lₜ, were L=the luminescence intensity in the presence of the compound.

The values of % cell versus a series of compound concentrations (0, 5, 10, 15, 20, 25, and 30 uM) were then plotted using Nonlinear regression analysis of Sigmoidal dose-response curve. IC₅₀ value was determined by the concentration causing a half-maximal percent activity.

IC₅₀ for example compounds, determined by the method above are given in the table below:

| | IC₅₀ for D425 cell | |
|---|---|---|
| Example | growth (μM) | Note |
| 1 | 5.6 | A = N |
| 2 | 9.4 | A = CH |

While typical embodiments have been set forth for the purpose of illustration, the foregoing descriptions and examples should not be deemed to be a limitation on the scope of the disclosure. Accordingly, various modifications, adaptations, and alternatives may occur to one skilled in the art without departing from the spirit and scope of the present disclosure.

We claim:

1. A compound of formula I: 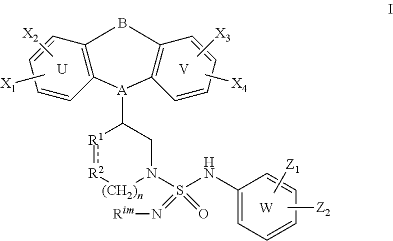
or a pharmaceutically acceptable salt thereof,
wherein:
A is N or CH
B is absent or is selected from a direct bond, —CH₂CH₂—, —CH═CH—, O, S, or —NR^{B}—C(O)—;
R¹ is selected from CH₂ and may also be —O— or NR^{Q} when A is CH only;
R² is selected from CH₂ or R¹ and R² taken together may for a carbon-carbon double bond;
R^{im} is selected from H or lower alkyl
U, V and W are independently cabocyclic aromatic or heteroaromatic rings;
n=0 or 1;
X₁, X₂, X₃ and X₄ are independently selected in each instance from hydrogen, halogen, nitro, cyano, (C₁-C₆)alkyl optionally substituted with —OH, (C₁-C₆)haloalkyl, (C₁-C₆)haloalkoxy, (C₁-C₆)haloalkylthio, —NR³R⁴, —OR³, —C(O)R³, —OC(O)R³, —C(O)NR³R⁴, —C(O)OR³, —SR³, —SO₂R³, and —SO₂NR³R⁴;
Z₁ and Z₂ are independently selected in each instance from hydrogen, halogen, nitro, cyano, azido, (C₁-C₆)alkyl, (C₁-C₆)haloalkyl, (C₁-C₆)haloalkoxy, (C₁-C₆)haloalkylthio, —NR³R⁴, —NR³C(O)R⁴, —NR³C(O)OR⁵, —OR³, —C(O)R³, —OC(O)R³, —C(O)NR³R⁴, —C(O)OR³, —SR³, —SO₂R³, —SO₂NR³R⁴, and five membered heterocyclyl;
R^{B} is selected from H, or lower alkyl
R^{Q} is selected from H, optionally substituted lower alkyl, aryl
R³, R⁴ and R⁵ are lower alkyl.

2. The compound of claim 1 where A is N.

3. The compound of claim 1 where A is CH.

4. The compound of claim any one of claims 1-3, wherein X₁, X₂, X₃ and X₄ are independently selected in each instance from hydrogen and halogen.

5. The comopound of any one of claims 1-4, wherein Z₁ and Z₂ are independently selected in each instance from hydrogen and (C₁-C₆)haloalkoxy.

6. The compound of any one of claims 1-5, wherein B is a direct bond or —CH₂CH₂—.

7. The compound of any one of claims 1-6, wherein R¹ and R² are both CH₂.

8. The compound of any one of claims 1-7, wherein R^{im} is H.

9. The compound of any one of claims 1-8, wherein n is 1.

10. The compound of claim 1, selected from 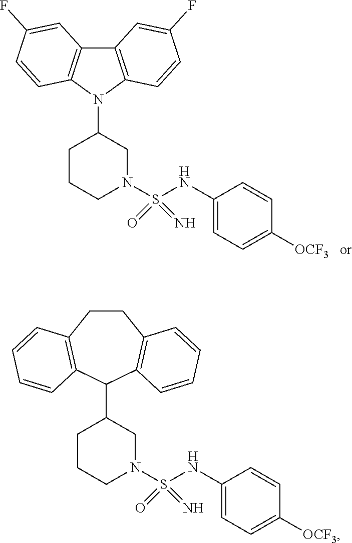
or a pharmaceutically acceptable salt thereof.

11. A composition, comprising the compound of any one of claims 1-10.

12. The composition of claim 11, which is a pharmaceutical composition further comprising at least one pharmaceutically acceptable carrier.

13. A method of treating a disease related to deficient Protein Phosphatase 2A (PP2A) activity in a subject, comprising administering the compound of any one of claims 1-10, or the composition of claim 11 or claim 12, to a subject in need thereof.

14. The method of claim 13, wherein the disease is a cancer, an inflammatory disease, an autoimmune disease, a fibrotic disease, or a neurodegenerative disease.

15. The method of claim 14, wherein the cancer is resistant to at least one previous therapy that was administered to the subject, and administration of the compound of any one of claims 1-10, or the composition of claim 11 or claim 12 resensitizes the cancer to said previous therapy.

16. The method of claim 13, wherein the disease is prostate cancer, breast cancer, lung fibrosis, or Alzheimer's disease.

17. A method of inhibiting a kinase, comprising contacting the kinase with the compound of one of claims 1-10, or the composition of claim 11 or claim 12.

18. The method of claim 17, which is an in vivo method.

19. The method of claim 17, which is an in vitro method.

20. An article of manufacture, comprising the compound of one of claims 1-10, or the composition of claim 11 or claim 12.
